(19) United States
(12) Patent Application Publication
(10) Pub. No.: US 2008/0027475 A1
(43) Pub. Date: Jan. 31, 2008

(54) PUNCTURING DEVICE WITH IMPEDANCE MEASURING FACILITY

(75) Inventor: Armin Grundmann, Reutlingen (DE)

Correspondence Address: BAKER & DANIELS LLP / ROCHE, 300 NORTH MERIDIAN STREET, SUITE 2700, INDIANAPOLIS, IN, 46204, US

(73) Assignee: ROCHE DIAGNOSTICS GMBH, Mannheim (DE)

(21) Appl. No.: 11/670,822

(22) Filed: Feb. 2, 2007

(30) Foreign Application Priority Data
Feb. 4, 2006 (EP) 06002329.8

(51) Int. Cl. A61B 17/34; A61B 5/04

(52) U.S. Cl. 606/185; 600/373

(57) ABSTRACT

A puncturing device for generating a puncturing wound for obtaining a body fluid sample of a human or animal, comprising a puncturing element drive in order to drive a puncturing element that is inserted into the puncturing device, an alternating voltage source designed to apply an alternating voltage signal with a rectangular leading edge to an electrode that is integrated into the puncturing element, an electrical measuring facility configured to measure a response signal elicited by applying the alternating voltage signal to the electrode, and an analytical facility configured to analyze the response signal and to control the puncturing element drive based upon a result of the analysis.

This application claims priority to EP 06002329.8, filed on Feb. 4, 2006, the disclosure of which is expressly incorporated herein by reference.

### FIELD OF THE INVENTION

The invention relates to a puncturing device for generating a puncturing wound for obtaining a body fluid sample of a human or animal, said device comprising a puncturing element drive for driving a puncturing element that is inserted into the puncturing device in order to perform a puncturing and returning motion, an alternating voltage source designed to apply an alternating voltage signal to an electrode that is integrated into an inserted puncturing element, an electrical measuring facility in order to measure a response signal elicited by the alternating voltage signal, and an analytical facility operative to determine an analytical result by analyzing the response signal and to control the puncturing element drive taking into consideration the analytical result.

The invention further relates to a puncturing system comprising a puncturing device of this type and a puncturing element for such a puncturing device of this type. The invention further relates to a method for determining the puncturing depth required for obtaining a body fluid sample of a human or animal.

### BACKGROUND OF THE INVENTION

A consistent goal in the development of puncturing devices for obtaining body fluid samples is the minimization of the pain associated with the puncture. In this context, the puncturing depth down to which the puncturing element is punctured into the skin of a patient is of particular relevance for the sensation of pain. Ideally, the puncturing depth should be no larger than absolutely necessary for obtaining a sample. Whereas no or an insufficient quantity of sample fluid is obtained if the puncturing depth is insufficient, a puncturing depth exceeding the required level leads to unnecessary pain.

For this reason, puncturing devices according to the state of the art commonly are provided with a setting facility that allows the puncturing depth to be set. Usually, optimal settings are determined on the basis of experience or by trial and error. Since the optimal puncturing depth depends on the thickness of the stratum corneum, values determined for a certain puncturing site of a certain patient cannot be applied to other puncturing sites on different parts of the body or, even more so, on other patients. For this reason, punctures made for obtaining body fluid samples are often excessively deep such that patients are caused pain that would be avoidable if the puncturing depth was set optimally.

Since the impedance of the stratum corneum differs from the impedance of skin layers underneath the stratum corneum and, in particular, from the impedance of body fluid, on principle an impedance measurement during the puncturing process can be used to determine whether or not the puncturing depth required for obtaining body fluids is reached. In the case of puncturing elements with integrated electrodes, wetting by body fluid leads to a change of the impedance being measurable which allows to conclude that the puncturing element has penetrated into the skin to the puncturing depth required for obtaining a body fluid sample. Puncturing devices with impedance measuring facilities and puncturing elements with integrated electrodes of this type are known, for example, from DE 19914485 C2 and WO 2004/080306 A1. In these devices, an impedance measuring facility is connected to a microprocessor that controls the puncturing element drive such that the puncturing element is retracted as soon as a change of impedance indicates that the puncturing depth required for obtaining a body fluid sample is reached.

Aside from the optimization of the puncturing depth, it is desirable to have a puncturing speed that is as high as possible in order to minimize the associated pain. However, the higher the speed at which the puncturing element is punctured into the skin, the more rapidly an impedance measurement has to be analyzed and the more rapidly a reversal of the direction of motion of the puncturing element has to be initiated by triggering the puncturing element drive. In order to provide for as rapid an analysis of the impedance measurement as possible, WO 2004/080306 A1 recommends the use of a sinusoidal alternating voltage with a frequency between 10 kHz and 1 MHz at a strength of current between 1 mA and 10 mA. However, it has become evident that sufficiently rapid response times for controlling puncturing processes with a duration of just a few milliseconds cannot be achieved by this means.

Presumably in order to address the issue of rapid analysis of an impedance measurement, DE 19914485 C2 recommends to advance the puncturing element gradually until a wetting of the puncturing element and thus the fact that a sufficient puncturing depth has been reached is determined by means of a change in impedance. However, gradual advancement of the puncturing element leads to the puncturing process taking longer and motions of the patient, in particular involuntary ones, possibly causing additional pain.

### SUMMARY OF THE INVENTION

The present invention relates to a way for determining more rapidly a wetting by body fluid of the puncturing element used for puncturing.

The present invention includes use of an alternating voltage source designed such that the alternating voltage signal has a rectangular leading edge, preferably it is a square wave voltage, and the leading edge has a voltage amplitude of 10 mV to 200 mV.

Upon the use of a square wave voltage, a characteristic change of impedance upon wetting of the puncturing element by body fluid showed up more clearly and more rapidly than upon the use of a sinusoidal voltage. As part of the invention it was found that, upon puncturing, a flank of a square wave voltage elicits a response signal that is characterized by a leading edge and a characteristic drop over the course of but a few microseconds. The use of a square wave voltage thus allows the analysis of the impedance measurement that is carried out according to the state of the art to be reduced to the analysis of a short response signal, preferably a pulse of current, that was elicited by a flank of the square wave voltage. By this means, a wetting of the electrode of a puncturing element can be recognized within but a few microseconds or even fractions of microseconds.

In one embodiment, the present invention employs a steep leading edge of a square wave voltage. However, a rectangular leading edge of this type is present not only in the case of square wave voltages in a narrower sense, in which the voltage switches periodically between exactly two voltage values. It is not detrimental to the purposes of the present invention if the employed voltage switches, for example, from a first extreme value (e.g. 50 mV) to a second value (e.g. 0 mV) after a first period of time D1 and then switches from this value to a second extreme value (e.g. −50 mV) after a second period of time D2 and then switches from this value back to the intermediate value (e.g. 0 mV) after a third period of time D3.

The invention further relates to a puncturing system comprising a puncturing device according to the invention and a replaceable puncturing element with an integrated electrode. Basically, any electrode material can be used for the integrated electrode, for example stainless steel or a platinum metal. In one embodiment, the present invention uses platinum black as electrode material for the integrated electrode. Platinum black may be obtained by electrolytic deposition of platinum and is characterized by its very porous and rough surface structure. Accordingly, the invention also relates to a puncturing element that comprises an integrated electrode with a coating made of platinum black. This is the case because it was found as part of the invention that lower transition resistances occur upon the use of puncturing elements of this type such that the measuring sensitivity can be improved.

The present invention also relates to a method for determining the puncturing depth of a puncturing element into a skin surface required for obtaining a body fluid sample of a human or animal, whereby an electrode that is integrated into the puncturing element is used to carry out an electrical measurement in order to determine whether the puncturing element is wetted by body fluid, characterized in that a square wave voltage is applied to the electrode for the purpose of the measurement.

As part of the invention, it has been found that even significantly lower strengths of currents than those between 1 mA and 10 mA that have been recommended in WO 2004/080306 A1 allow meaningful measurements to be carried out. For this reason, it is preferable to match the alternating voltage source and the electrode of the puncturing element to each other such that an alternating current with a current amplitude of no more than 500 μA, particularly preferred of no more than 200 μA, flows during puncturing. In this context, current amplitude shall be defined as the peak value of the current response that is elicited by a flank of the square wave voltage. Preferably, the square wave voltage used has a voltage amplitude of 10 mV to 200 mV, particularly preferably of 10 mV to 100 mV, in particular of 20 mV to 70 mV. In this context, the voltage amplitude shall be defined as half of the value of the change of voltage occurring at a flank of the square wave voltage.

Further details of the invention shall be illustrated in the following based on one exemplary embodiment with reference to the appended drawings. The particularities presented therein can be used individually or in combination to create preferred further developments of the invention. In the figures:

### BRIEF DESCRIPTION OF THE DRAWINGS

FIG. 1 shows a schematic view of a puncturing element with integrated electrode in the process of penetrating the skin;

FIG. 2 shows a schematic view of a puncturing device according to the invention;

FIG. 3 shows an example of the time course of a square wave voltage;

FIG. 4 shows an example of a response signal measured upon penetration of a puncturing element into the skin; and

FIG. 5 shows an example of an equivalent circuit diagram of a measurement carried out according to the invention.

### DETAILED DESCRIPTION OF EMBODIMENTS OF THE INVENTION

The embodiments disclosed below are not intended to be exhaustive or to limit the invention to the precise forms disclosed in the following detailed description. Rather, the embodiments are chosen and described so that others skilled in the art may utilize their teachings.

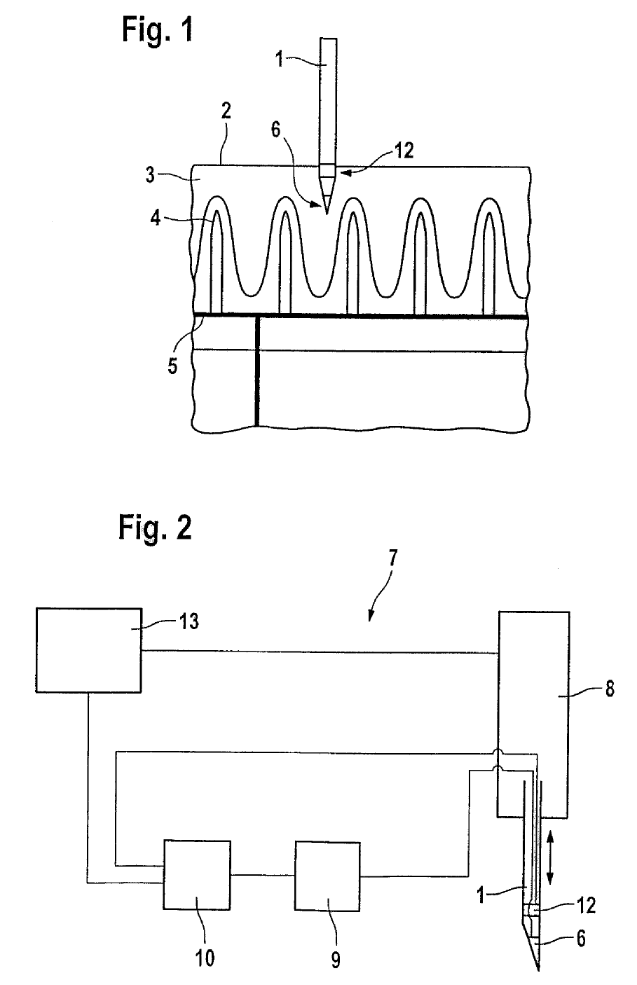

FIG. 1 shows a schematic cross-sectional view of the structure of the skin and the relationships upon puncturing with a puncturing element 1. The surface of the skin is formed by the stratum corneum 2 underneath which there is a capillary layer 3 with capillary loops 4. Blood-storing venules 5 supply blood to the capillary loops 4.

Whereas the electrical conductivity, for example, of the upper layers of the skin below the stratum corneum at measuring frequencies of less than 100 kHz is only approx. 2 mS/cm, the electrical conductivity of body fluids, such as, for example, blood, interstitial fluid or blood plasma, is at least twice as high such that an electrical measurement can be used to determine whether or not an electrode 6 integrated into a puncturing element 1 is wetted by body fluid and thus to determine whether or not a sufficient puncturing depth for obtaining a body fluid sample has been reached. The conductivity of the stratum corneum is significantly lower than the conductivity of the underlying skin layers.

The puncturing element 1 shown in FIG. 1 is part of a puncturing system that comprises, aside from replaceable puncturing elements 1, a puncturing device into which the puncturing elements 1 can be inserted. The structure, on principle, of the puncturing device with a puncturing element 1 inserted is shown schematically in FIG. 2. The puncturing device 7 comprises a puncturing element drive 8 for driving a puncturing element 1 that is inserted into the puncturing device 7 for a puncturing and returning motion, an alternating voltage source 9 that is designed to apply an alternating voltage signal to the electrode 6 of an inserted puncturing element 1, and an electrical measuring facility 10 for measuring a response signal elicited by the alternating voltage signal.

The puncturing device 7 can further comprise an electrode, in particular an annular electrode, which, according to its purpose, touches the skin upon puncturing and forms the counter-electrode for the electrode 6 of a puncturing element 1. However, the puncturing device is preferably provided for use with puncturing elements 1 that comprise a counter-electrode 12 for the electrode 6. A bipolar structure of this type allows for improved measuring sensitivity in that all of the length measured between the electrode 6 and the counter-electrode 12 can be wetted.

The puncturing device 7 further contains an analytical facility 13 in the form of a microprocessor, which, when operative, determines an analytical result by analyzing the response signal and controls the puncturing element drive 8 taking into consideration the analytical result. This means allows the return motion for retraction of the puncturing element 1 to be initiated as soon as a wetting of the electrode 6 that is integrated into the puncturing element 1 is detected, and thus allows the puncturing depth to be minimized to the extent required for obtaining the sample in order to minimize the pain.

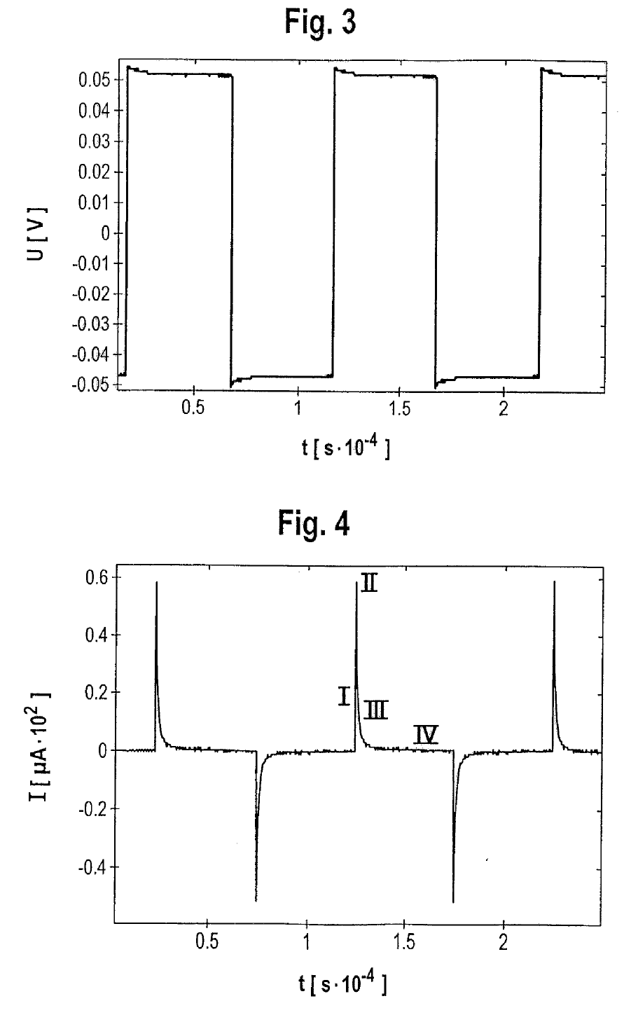

One feature of the puncturing device 7 shown is that the alternating voltage source 9 generates a square wave voltage as alternating voltage signal. FIG. 3 shows an example of the time course of a suitable square wave voltage. In this context, the time t is shown in units of 10⁻⁴ seconds on the abscissa and the voltage U in units of Volt is shown on the ordinate. The square wave voltage preferably has a voltage amplitude of less than 200 mV, particularly preferably of 10 mV to 100 mV, particularly of 20 mV to 60 mV. The square wave voltage shown in FIG. 2 has a voltage amplitude of 50 mV and a frequency of approx. 100 kHz. The frequency of the square wave voltage used should be at least 10 kHz. Well-suited for this purpose are frequencies of 10 kHz to 1 MHz, in particular 10 kHz to 500 kHz. Particularly well-suited for this purpose are frequencies of 50 kHz to 100 kHz.

The alternating voltage signal shown in FIG. 3 can be used with the set-up shown in FIG. 2 to measure the electrical impedance between the electrodes 6 and 12 that are integrated into the puncturing element 1. FIG. 4 shows in an exemplary fashion the response signal that is elicited by the alternating voltage signal in a measurement of this type. The strength of the current I in units of μA flowing between the electrodes 6, 12 is plotted over time t in units of 10⁻⁴ s as response signal. However, the dropping voltage between the two electrodes 6, 12 can obviously also be considered as the response signal.

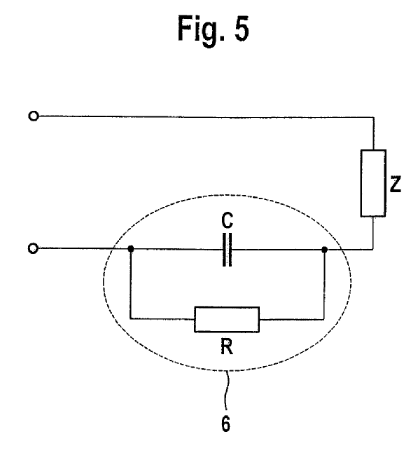

The course of the response signal shown can be subdivided into four typical phases, I, II, III, IV, that shall be illustrated in the following with reference to the equivalent circuit diagram shown in FIG. 5. FIG. 5 shows the electrode 6 of the puncturing element 1 in a simplified fashion as a parallel circuit made up by a capacitance C and an ohmic resistance R. Tissue and/or body fluid being between the electrode 6 of the puncturing element 1 and the electrode 12 of the puncturing device 7 effects the impedance Z shown in FIG. 5.

Phase I in FIG. 4 is characterized by an abrupt increase of the flow of current. The start of phase I coincides with one flank of the square wave voltage. In the case of an ideal square wave voltage with an infinitely steep flank, the duration of phase I is infinitely short. However, ideal square wave voltages occurring in accordance with the exemplary view of FIG. 3 do not have an infinitely steep flank such that the resulting duration of phase I is finite, although very short.

Phase I leads into phase II in which a peak current elicited by one flank of the square wave voltage occurs. According to the equivalent circuit diagram shown in FIG. 5, the amplitude of the peak current essentially depends only on the impedance Z of the tissue and/or body fluid between the electrodes if one neglects any ohmic resistances of various leads. This is namely because the resistance R shown in FIG. 5 that is based on the existence of a boundary layer between the electrode and surrounding tissue and/or body fluid, is parallel to the capacitance C. For this reason, even just an analysis of the peak current occurring in phase II can be used to determine whether or not the electrode of the puncturing element 1 is wetted by body fluid and thus to determine whether or not a puncturing depth sufficient for obtaining a body fluid sample is reached.

After phase II follows a phase III in which the capacitance C formed by the electrode discharges via the resistance R such that there is an exponential drop of the strength of current with the characteristics of an RC component. The resistance R that is relevant for the discharge of the capacitance C is based mainly on the existence of the boundary layer between the electrode and surrounding tissue and/or body fluid such that, on principle, an analysis of phase III can also be used to determine whether the electrode is wetted by body fluid.

Regarding the response signal shown in FIG. 5, after phase III follows a phase IV in which no current flows any longer. The duration of phase IV mainly depends on the frequency of the square wave voltage. Phase IV may disappear if high frequencies are used.

Preferably, the electrode 6 of the puncturing element 1 and the alternating voltage source 9 of the puncturing device 7 are matched to each other such that upon wetting of the puncturing element 1 by body fluid the peak current of the response signal elicited by the flank of the square wave voltage drops exponentially to less than half of its value within 1 μs. It is particularly favorable for the peak current to drop within 1 μs to less than 50 μA, preferably to less than 30 μA, in particular to less than 20 μA.

The electrode 6 of the puncturing element 1 having a coating made of platinum black provides for a low transition resistance of the electrode such that a wetting can be detected more easily. Preferably, the counter-electrode 12 that is integrated into the puncturing element 1 also has a coating made of platinum black.

In one embodiment, to detect a wetting of the electrode 6 of the puncturing element 1 by body fluid as early and as reliably as possible the electrode surface area of the electrode 6 is less than 10000 μm², preferably less than 6000 μm², particularly preferably less than 4000 μm², and in particular less than 3000 μm². Preferably, the electrode surface area of the counter-electrode 12 also is less than 10000 μm², particularly preferably less than 6000 μm², in particular less than 4000 μm². Small electrode surface areas minimize interfering influences. A puncturing element whose electrodes 6 and 12 have surface areas of approx. 2900 μm² and 2600 μm², respectively, were used in the measurements shown in FIG. 4.

The electrodes 6 and 12 are arranged on the puncturing element 1 with a distance of several 10 μm. An electrode distance of not more than 100 μm is advantageous, especially an electrode distance of 20 μm to 30 μm.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. Puncturing device for generating a puncturing wound for obtaining a body fluid sample of a human or animal, comprising
a puncturing element drive for driving a puncturing element that is inserted into the puncturing device in order to perform a puncturing and returning motion,
an alternating voltage source designed to apply an alternating voltage signal to an electrode that is integrated into the inserted puncturing element,
an electrical measuring facility configured to measure a response signal elicited by the application of the alternating voltage signal to the electrode, and
an analytical facility configured to analyze the response signal and to control the puncturing element drive based upon a result of the analysis, wherein
the alternating voltage signal has a rectangular leading edge with a voltage amplitude of between 10 mV and 200 mV.

2. Puncturing device according to claim 1, wherein the alternating voltage signal is a square wave voltage.

3. Puncturing device according to claim 1, wherein the voltage amplitude is between 0 mV and 100 mV.

4. Puncturing device according to claim 2, wherein the square wave voltage has a frequency of between 10 kHz and 1 MHz.

5. Puncturing device according to claim 1, wherein the electrode is a counter-electrode.

6. Puncturing system comprising
a puncturing device generating a puncturing wound for obtaining a body fluid sample of a human or animal, comprising
a puncturing element drive configured to perform a puncturing and returning motion,
an alternating voltage source designed to apply an alternating voltage signal having a rectangular leading edge with a voltage amplitude of between 10 mV and 200 mV,
an electrical measuring facility configured to measure a response signal elicited by the application of the alternating voltage signal, and
an analytical facility configured to analyze the response signal and to control the puncturing element drive based upon a result the analysis, and
a replaceable puncturing element with an integrated electrode, wherein the puncturing element is configured for insertion into the puncturing device, the
alternating voltage signal is applied to the electrode, and the response signal is elicited by application of the alternating voltage signal to the electrode.

7. Puncturing system according to claim 6, wherein the replaceable puncturing element comprises a counter-electrode for the electrode.

8. Puncturing system according to claim 6, wherein the alternating voltage source and the electrode are matched to each other such that an alternating current with a current amplitude of no more than 500 μA, flows during puncturing.

9. Puncturing system according to claim 6, wherein the alternating voltage source is a square wave voltage, and the electrode and the alternating voltage source are matched to each other such that upon wetting of the puncturing element by body fluid a peak value of the response signal elicited by a flank of the square wave voltage drops to less than half of its value before wetting within 1 μs.

10. Puncturing system according to claim 6, wherein the electrode includes a coating made of platinum black.

11. Puncturing system according to claim 10, wherein the electrode has a surface area of less than 10000 μm².

12. Method for determining the puncturing depth of a puncturing element required for obtaining a body fluid sample of a human or animal, including the steps of:
applying a square wave voltage to an electrode that is integrated into the puncturing element;
performing to an electrical measurement on the electrode in order to determine whether the puncturing element is wetted by body fluid.

13. Puncturing device according to claim 3, wherein the voltage amplitude is between 20 mV and 70 mV.

14. Puncturing device according to claim 4, wherein the frequency is between 10 kHz and 500 kHz.

15. Puncturing device according to claim 4, wherein the frequency is between 50 kHz and 100 kHz,

16. Puncturing system according to claim 8, wherein the current amplitude is less than 200 μA.

17. Puncturing system according to claim 11, wherein the surface area is less than 6000 μm².

18. Puncturing system according to claim 11, wherein the surface area is less than 4000 μm².

19. Puncturing system according to claim 11, wherein the surface area is less than 3000 μm².
